(19) United States
(12) Patent Application Publication
(10) Pub. No.: US 2026/0347376 A1
(43) Pub. Date: Nov. 5, 2026

(54) PRECURSOR SOLUTION SUITABLE FOR THE PREPARATION OF HIGH PERFORMANCE EPITAXIAL REBa2Cu3O7-x SUPERCONDUCTORS

(71) Applicant: CONSEJO SUPERIOR DE INVESTIGACIONES CIENTÍFICAS (CSIC), MADRID (ES)

(72) Inventors: MARÍA TERESA PUIG MOLINA, CERDANYOLA DEL VALLÈS (ES); FRANCESC XAVIER OBRADORS BERENGUER, CERDANYOLA DEL VALLÈS (ES); SUSANA RICART MIRO, CERDANYOLA DEL VALLÈS (ES); LAVINIA SALTARELLI, CERDANYOLA DEL VALLÈS (ES); DIANA GARCIA FRANCO, CERDANYOLA DEL VALLÈS (ES); KAPIL GUPTA, CERDANYOLA DEL VALLÈS (ES)

(21) Appl. No.: 19/099,311

(22) PCT Filed: Jul. 28, 2023
(86) PCT No.: PCT/EP2023/070988

§ 371 Date: Jan. 28, 2025

(30) Foreign Application Priority Data
Jul. 29, 2022 (EP) 22382741.1

(51) Int. Cl. C07F 5/00; C30B 19/00; C30B 29/22

(52) U.S. Cl. CPC C07F 5/003; C30B 19/00; C30B 29/225

(57) ABSTRACT

A REBCO precursor solution including: a propionate salts of Re, Ba; of Cu; an amine; and a solvent system; where: the metals RE, Ba, and Cu are found exclusively in the propionate salts, which are the only salts present in the solution. The total metal concentration is 1-2 M of the solution. The solvent system of the solution is an alcohol (C₁-C₄): propionic acid mixture in a ratio of 20:80-60:40. The amine is an amine which is soluble in the solvent system and is present in a concentration between 1-8% in volume of the total volume of the solution. The molar ratio of amine:copper in the solution is a 0.3:1-2:1 ratio and the solution is free of F and free of acetate. solution can include or be in the form of nanoparticles and is useful for the preparation of superconducting REBCO (or YBCO) layers.

This application claims the benefit of European Patent Application EP22382741 filed on 29 Jul. 2022.

### TECHNICAL FIELD

The invention relates to the field of high-temperature superconducting materials, and in particular, to REBa₂Cu₃O₇₋ₓ (RE=rare earth or Y) precursor solutions for the preparation of such superconductor materials.

### BACKGROUND ART

The development of alternative energy sources has experienced an exponential progress in the past decades, given the necessity of reducing the environmental impact of years of fossil fuels energy sources exploitation. Superconducting materials opened new opportunities to solve the problem of efficient electricity transport, as they uniquely display no losses in large current transport. The discovery of High-Temperature Superconductors (HTS) pushed forward this technology, although many difficulties had to be overcome.

Nowadays, HTS are promising candidates for various applications, not only in clean power energy devices, such as power cables, fault current limiters, transformers, but also for equipment working at ultra-high magnetic fields (accelerators, NMR, fusion reactors) and transport (ships, levitating trains, electrical airplanes).

Successive to the discovery of HTS, the widespread of superconductivity was delayed by the need to develop fabrication methods of superconducting REBa₂Cu₃O₇₋ₓ (REBCO materials) where RE is Yttrium or a rare earth element and x is <0.1. The introduction of HTS on a flexible tape architecture that allow the industrial implementation, specifically Coated Conductors (CCs), boosted their production spread, with successful production of hundreds-of-meters-long pieces with current capacities in the range 200-700 A per 1 cm width of tape. CCs required the implementation of a thin film technology to a km-length application, being epitaxial multilayers deposited on a long length flexible metallic substrate. However, the production costs of the available technology are still too high. The techniques commonly employed for the growth of the HTS layer are Pulsed Laser Deposition (PLD), Metal Organic Chemical Vapour Deposition (MOCVD), Evaporation, trifluoroacetate metal-organic decomposition—Chemical Solution Deposition (TFA-CSD), which mostly imply expensive vacuum equipment and/or slow diffusion kinetics.

A recent approach involved in low-cost fabrication of YBCO superconductors, is the Transient Liquid Assisted Growth (TLAG), a non-equilibrium process based on chemical methods that enables growth rates more than two order of magnitude larger than the existing technologies (see L. Soler et al.; Nat. Commun., 2020, vol. 11, p. 344). It is based on the formation of a transient liquid of Ba—Cu—O that incorporates dissolved Y ions that, upon a fast atomic diffusion process towards the substrate growth front, kinetically governs the epitaxial growth of the YBCO layers. For that purpose, environmentally friendly non-fluorinated precursors for the preparation of YBCO solutions were developed, despite the need to overcome the elimination of BaCO₃ at rather low temperatures (an intermediate compound in YBCO formation, its elimination being the limiting step of the reaction) (cf. P. Vermeir et al.; “Elucidation of the Mechanism in Fluorine-Free prepared YBa₂Cu₃O_{7-δ} Coatings” Inorg. Chem., 2010, vol. 49, pp. 4471-4477).

The use of acetates of yttrium, barium, and copper as precursors for the preparation of fluorine-free YBCO precursor solutions, through dissolution in HProp-based media is known in the art (cf. P. Vermeir et al., “Influence of sintering conditions in the preparation of acetate-based fluorine-free CSD YBCO films using a direct sintering method”; Mater. Res. Bull., 2012, vol. 47, pp. 4376-4382; and Yue Zhao et al; “Growth of Highly Epitaxial YBa₂Cu₃O_{7-δ} Films from a Simple Propionate-Based Solution”, Inorg. Chem. 2015, vol. 54, pp. 10232-10238). However, complete conversion of acetates into propionates is never assured; specifically, in the case of barium acetate (see S. Rasi et al.; “Relevance of the Formation of Intermediate Non-Equilibrium Phases in YBaCuO im Growth by Transient Liquid Assisted Growth”; The Journal of Physical Chemistry 2020 vol. 124; pp. 15574-15584). The presence of product mixtures in solution in particular the presence of acetates may endure different decomposition paths, hindering an optimal and reproducible result of the final pyrolyzed films.

M. Nasui et al discloses a REBCO solution free of F that is obtained mixing previously prepared solutions of the propionates of Y, Ba, and Cu and by adding a chelating agent (glycerol). The corresponding metal acetates are used as reagents to prepare such solutions of the propionates of Y, Ba, and Cu and, therefore, the final REBCO solution still may include Ba acetate trace (see M. Nasui et al; “Fluorine-fee propionate route for the chemical solution deposition of YBa₂Cu₃O₇₋ₓ superconducting films”; Ceramics International 2015, vol. 41, pp. 4416-4421). In this case, also the elimination of the acetates present in the propionate solution is not ensured.

L. Soler et al mentioned above discloses that by using fluorine-free solutions of YBCO using a transient liquid-assisted growth process combined with chemical solution deposition, fabrication of YBCO layers can be attained at growth rates beyond 100 nm/s. The solution of YBCO is prepared from the corresponding acetate salts at a concentration of 1.5 M and using triethylamine. (cf. L. Soler et al.; “Ultrafast transient liquid assisted growth of high current density superconducting films”; Nat. Commun., 2020, vol. 11, p. 344).

Finally, CN106242553A discloses the preparation of fluorine-free precursor solution by dissolving rare earth (RE) propionate, barium propionate, and copper propionate in a mixed solvent of propionic acid and ethanol according to the atomic ratio of RE:Ba:Cu=1:1.75:3 at a temperature between 40 °C-100 °C yielding the fluorine-free precursor solution with a total cation molar concentration of 1.5 mol/L. This document teaches that the BaCO₃ generated can be eliminated by introducing some fluorine precursor, thus making this route a fluorine-based route which is out of the scope of the present patent.

However, despite what is disclosed in the art, there is still the need of providing stable solutions of yttrium, barium, and copper propionates for TLAG-CSD growth of epitaxial YBCO superconducting films of high thickness, and which are free of residual acetates as a consequence of the inefficient transformation of the original acetate salts to propionate salts of the YBCO precursor solutions.

### SUMMARY OF INVENTION

Inventors of the present invention have developed a novel class of fluorine-free solutions based on fluorine-free pure metal-propionates of yttrium, barium, and copper, through the introduction of an amine such as monoethanolamine (MEA) as an additive, which are suitable for transient liquid assisted growth-chemical solution deposition (TLAG-CSD) growth of epitaxial REBCO superconducting films of high thickness with reproducible results. Since they are fluorine free solutions, they meet the requirements of green chemistry. The use of an amine is advantageous both in increasing the solubility, aiding the dissolution of Cu(Prop)₂ through the formation of a Cu-amine complex, as well as increasing the thickness of the pyrolyzed layers with respect to the case of solutions in which an amine is not employed. The selection of the amine and of the amine content contributed to obtain nanoscale homogeneous pyrolyzed layers with the necessary characteristics for TLAG. Besides, stable solutions are reached, achieving chemical and microstructural nanoscale homogeneity for pyrolyzed films for their further TLAG growth.

Thus, a first aspect of the present invention relates to a REBCO precursor solution comprising: a propionate salt of RE; a propionate salt of Ba; a propionate salt of Cu; an amine; and a solvent system; wherein: RE means either Y or an earth metal, and the metals RE, Ba, and Cu are exclusively found in the form of propionate salts; the propionate salts are the only salts present in the solution; the metals are in a total metal molar concentration in a range of from 1-2 M of the solution; the solvent system is an alcohol (C₁-C₄):propionic (Hprop) acid mixture in a volume ratio of 20:80-60:40; the amine is an amine which is miscible in the solvent system at room temperature and is present in a concentration between 1-8% in volume of the total volume of the solution; the molar ratio amine:copper is of 0.3:1-2:1, and the solution is free of F and free of acetate.

The process employed to synthesize propionates of yttrium, barium, and copper, successfully eliminate the possibility of mixtures of products deriving from the incomplete conversion of acetate precursors in the solvent mixture by a robust, and scalable process which takes place with high-performance. Moreover, the synthetic process is easy to be carried out, resulting in high purity products with high yields and, most importantly, being extremely cost-effective as compared with commercially available acetate precursors.

Thus, a second aspect of the present invention relates to a process for preparing the REBCO precursor solution as defined above, comprising the steps of: a) providing a mixture of (C₁-C₄) alcohol:propionic acid in a volume ratio in the range of from 20:80 to 60:40; b) adding barium propionate, copper propionate, and RE propionate to the mixture of solvents in a consecutively manner, wherein each propionate salt is added once the previous propionate salt has been dissolved, and wherein the amount of propionate salts is an appropriate amount to obtain a total sum of metals concentration of the propionate salts in a range of from 1-2 M; and c) adding an amine in a concentration between 1-8% in volume of the total volume of the solution, wherein the molar ratio amine:copper is in the range of form 0.3:1 to 2:1.

The REBCO solution allows the obtaining of homogenous layers as well as the preparation of multi-layered superconducting films on a substrate. Ultrafast growth rates between 100 nm/s-2500 nm/s, using transient liquids during the epitaxial growth of the superconducting layer in the framework of TLAG are obtained. The TLAG-CSD process used is based on stable solution of RE (Y), Ba, and Cu propionates in the adequate stoichiometry. The stability of the solution allows to use high performance printing deposition methods such as ink-jet printing or slot-die coating. Moreover, the use of an amine additive in the precursor solution allows for high concentrations to be obtained resulting in films of thickness up to 2.5 μm through multideposition without hindering the highly homogeneous outcome of the pyrolysis process and favouring the epitaxial growth through TLAG process. The thickness is related to the solution concentration and viscosity.

Accordingly, a third aspect of the present invention is the use of the REBCO precursor solution for the preparation of superconducting REBCO layers of 0.1-2.5 μm thickness.

Another aspect of the present invention relates to a process for preparing superconductors based on growing superconducting REBCO layers of 0.1-2.5 μm thickness, the process comprising the following steps: a) disposing the precursor solution as defined above onto a surface of an appropriate substrate, for example metal with deposited biaxially textured oxide layers (buffer layers) or a monocrystal, by means of any method enabling the homogeneous control of the thickness of the film to form a precursor film; b) submitting the precursor film to a pyrolysis process by means of a thermal treatment in a controlled atmosphere; c) optionally repeating the steps a) to b) to obtain thicker pyrolyzed films by a multideposition process; d) submitting the deposited REBCO layer of step b) or of the REBCO multilayer of step c) to a process of TLAG for the growth of the final epitaxial REBCO layers; and e) submitting the superconductor thus obtained to an oxygenation process. Thus, the superconductor according to the invention is multideposited superconducting layers on a metallic substrate with biaxially textured oxide layers.

Finally, another aspect of the present invention relates to a multideposited superconducting layer on an appropriate substrate, for instance a biaxially textured substrate, obtainable by the process described above. This multi-conductor multilayer substrate is an epitaxial cuprate superconducting films, more precisely, high performance REBCO superconducting layers of thickness beyond 500 nm with a critical current density of 2-4 MA/cm² at 77K, in particular of 2.5 MA/cm² at 77K, thus being a high-performance REBCO superconducting layer.

### DESCRIPTION OF DRAWINGS

FIG. 1. is an ATR FT-IR of Cu(Prop)₂.

FIG. 2 is a high resolution XRD of CuProp₂.

FIG. 3 is an ATR FT-IR of BaProp₂.

FIG. 4 is a high resolution XRD of BaProp₂.

FIG. 5 is an ATR FT-IR of YProp₃.

FIG. 7 is an ATR FT-IR of GdProp₃.

FIG. 8 is a high resolution XRD of GdProp₃.

FIG. 9(a) shows the viscosity measurements with varying quantity of MEA for (3:7) solutions of 1 M and 1.75 M concentrations. FIG. 9(b) shows the evolution of H₂O wt % over time for (3:7) solutions with and without MEA, measured using Karl-Fischer titration method.

FIG. 10 shows optical microscopy (OM) images and XRD patterns of pyrolyzed samples with 2 layers of (a, b) (2:3) composition (a YBCO-stoichiometric mixture with a Y—Ba—Cu proportion of 1:2:3) using 1.75 M+4.3% v/v MEA solution; (c, d) (3:7) composition (a Cu-rich mixture with proportion of Y—Ba—Cu of 1:2:4.66) using 1.75 M+4% v/v MEA solution; (e, f) (4:11) composition (an excess Cu-rich mixture with a proportion of Y—Ba—Cu of 1:2:5.5) using 1.75 M+4% v/v MEA solution; and 1 layer of (g, h) (3:7) composition using excess MEA, in this case, 1.75 M+8% v/v MEA solution, respectively.

FIG. 11 shows a cross-sectional low-magnification STEM-HAADF images of (3:7) as-pyrolyzed thin films deposited using 1.75 M+4% v/v MEA solution with (a) 2 layers, (b) 4 layers, and (c) 8 layers. Magnified STEM-HAADF images displaying similar thicknesses of individual layers for (d) 2 layers, (e) 4 layers, (f) 8 layers. (g-i). The pore density analysis using Image J from the red rectangular regions is shown in (d-f), respectively, where pores are coloured in red for quantification. Histograms of pore area are used to calculate the average pore sizes.

FIG. 12 (a) shows a STEM-HAADF image of a two layer (3:7) pyrolyzed film deposited using 1.75 M+4% v/v MEA solution. Elemental EELS maps of (b) Cu-L edge, (c) Ba-M edge, (d) O-K edge, (e) composite EELS map of Cu (blue) and Ba (yellow), from red rectangular region in (a). (f-h) HR-TEM images collected from different regions of the pyrolyzed film, displaying the individual sizes and the presence of all precursor phases in the same area, which exhibit a homogeneous distribution.

FIG. 13 is a STEM-EDX cross-sectional elemental maps of Cu, Y, and Ba.

FIG. 14 is a TEM image of a) BaZrO₃ nanoparticles redispersed in ethanol b) BaZrO₃ nanoparticles in YBCO nanocomposite precursor solution c) Solution XRD of BaZrO₃ nanoparticles redispersed in YBCO precursor solvents of MEA, ethanol, methanol and propionic acid In TEM images.

### DETAILED DESCRIPTION OF THE INVENTION

All terms as used herein in this application, unless otherwise stated, shall be understood in their ordinary meaning as known in the art. Other more specific definitions terms as used in the present application are as set forth below and are intended to apply uniformly throughout the specification and claims unless an otherwise expressly set out definition provides a broader definition.

The term “room temperature” refers to a temperature of about 20 °C to about 25 °C.

The term “pyrolysis” and the term “carbonization” have been used herein indistinctively.

The term “REBCO” includes “YBCO”, when RE is used it refers always to both earth element and Y. REBa₂Cu₃O₇₋ₓ also named REBCO materials are those materials where RE is Ytrium (Y) or a rare earth element and x is <0.1.

The preparation of REBCO, in particular YBCO precursor solutions, follows the same procedure independently of the stoichiometries of the solution. The various stoichiometries of solutions differ in the RE-Ba—Cu molar ratio, and are named considering the Ba—Cu molar ratio of the transient liquid formed during the transient liquid assisted growth (TLAG) process, as following: the REBCO-stoichiometric mixture with a RE-Ba—Cu molar ratio of 1:2:3 ((2:3) composition); a Cu-rich mixture with proportion of RE-Ba—Cu of 1:2:4.66 ((3:7) composition); excess of Cu-rich composition corresponding to a molar ratio of RE-Ba—Cu of 1:2:5.5 ((4:11) composition).

It is noted that, as used in this specification and the appended claims, the singular forms “a”, “an”, and “the” include plural referents unless the context clearly dictates otherwise.

The REBCO, in particular, YBCO precursor solution of the present invention comprises: a propionate salt of RE (Y); a propionate salt of Ba; a propionate salt of Cu; an amine; and a solvent system; wherein: the metals RE (Y), Ba, and Cu are exclusively found in the form of propionate salts; the metals are in a total metal concentration in the range between 1-2 M of the solution; the solvent system is an alcohol (C₁-C₄):propionic (Hprop) acid mixture in a volume ratio range of from 20:80 to 60:40; the amine is an amine which is miscible in the solvent system and is present in a concentration between 1-8% in volume of the total volume of the solution; the molar ratio amine:copper is of 0.3:1-2:1, and the solution is free of F and free of acetate. In these precursor solutions, the propionate salts are the only salts present in the solution. The copper and the amine are partially coordinated as it is seen by Infrared analysis (IR) and by electronic paramagnetic resonance (EPR).

In a particular embodiment, the metals are in a total metal concentration in the solution in the range between 1-1.5 M. In another particular embodiment, the metals are in a total metal concentration in the solution in a concentration of 1.5 M.

In another particular embodiment of the present invention, the REBCO precursor solution is that where RE is selected from Gadolinium or yttrium.

Appropriate rare earth metals for use in the present invention are, neodymium (Nd), Samarium (Sm), Europium (Eu), gadolinium (Gd), Terbium (Tb), Dysprosium (Dy), Holmium (Ho), Erbium (Er), Thulium (Tm), Ytterbium (Yb), and Lutetium (Lu). Preferably the RE metal is yttrium or gadolinium.

In another particular embodiment of the present invention, the REBCO precursor solution is that which is selected from the group consisting of:
a) a REBCO-stoichiometric mixture with a molar ratio of Y:Ba:Cu of 1:2:3;
b) a Cu-rich mixture with molar ratio of RE:Ba:Cu of 1:2:4.66;
c) an excess Cu-rich mixture with a molar ratio of RE:Ba:Cu of 1:2:5.5; and
d) a Y-rich and excess of Cu-rich mixture with a molar ratio of RE:Ba:Cu of 1.35:2:5.5.

Preferably the RE is yttrium (Y).

In a particular embodiment of the present invention, the REBCO precursor solution is that which is a Cu-rich mixture with molar ratio of RE:Ba:Cu of 1:2:4.66. This solution works especially well enhancing epitaxial growth of REBCO in TLAG.

Any of the above embodiments with RE=Y is a particular embodiment of the invention.

The amine forms a complex with Cu(Prop)₂ and, aids in increasing Cu(Prop)₂ solubility in the media used. It also contributes to increase the stability of the solution and increases the final film thickness. During the pyrolysis process the propionates decompose and constitute part of the intricate metalorganic skeleton in a way to properly release the strain during the pyrolysis process avoiding cracking. In a particular embodiment, the REBCO precursor solution according to the invention is that where the amine is selected from the group consisting of (C₁-C₄)-alcoholamine, secondary at tertiary amines as catecholamine, and pyridine. Examples of catecholamines are epinephrine (adrenaline), norepinephrine (noradrenaline), and dopamine. Examples of C₁-C₄)-alcoholamines are methanolamine and ethanolamine. In another particular embodiment, the amine contains at least an —OH functional group together with —NH₂ functional group bearing possible stabilization through H-bonds, apart from feasibility for complexation. In another particular embodiment, the amine is a (C₁-C₄)-alcoholamine. In another particular embodiment of the present invention, the REBCO precursor solution is that where the amine is selected from the group consisting of methanolamine, ethanolamine, a catecholamine, and pyridine. In another particular embodiment of the present invention, the REBCO precursor solution is that where the amine is selected from the group consisting of (C₁-C₄)-alcoholamine, catecholamine, triethylamine, lutidine, 4-formylpyridine, 2-acetylpyridine, and pyridine. In another particular embodiment of the present invention, the REBCO precursor solution is that where the amine is selected from the group consisting of (C₁-C₄)-alcoholamine, triethylamine, pyridine, lutidine, 4-formylpyridine, and 2-acetylpyridine. In a preferred embodiment, the amine is monoethanolamine (MEA).

In another particular embodiment, the REBCO precursor solution is that where the concentration of the amine in the solution is between 1-5% in volume of the total volume of the solution. In another particular embodiment, the REBCO precursor solution is that wherein the concentration of the amine in the solution is between 1-4 in volume of the total volume of the solution. In another particular embodiment, the REBCO precursor solution is that wherein the concentration of the amine in the solution is between 2-4% in volume of the total volume of the solution. In another particular embodiment, the REBCO precursor solution is that wherein the concentration of the amine in the solution is 4% in volume of the total volume of the solution. With this percentage an especially stable solution and a high quality pyrolyzed films of 450 nm in thickness can be obtained in combination with a metal concentration in the solution of 1.75 M for instance, when the deposition technique is spin coating.

In another particular embodiment of the present invention, the REBCO precursor solution and in particular the REBCO precursor solution is that wherein the molar ratio amine: copper is 0.5:1 to 2:1. In another particular embodiment of the present invention, the REBCO precursor solution is that wherein the weight ratio amine:copper is 0.6:1.

In a particular embodiment of the present invention, the amine is methanolamine and the viscosity of the REBCO solution is in a range comprised from 3 to 25 mPa·s. In another particular embodiment, the viscosity of the REBCO solution is in a range comprised from 4 to 15 mPa·s. In another particular embodiment, the viscosity of the REBCO solution is in a range comprised from 4 to 5 mPa·s.

In another particular embodiment of the present invention, the REBCO precursor solution is that wherein the alcohol (C₁-C₄) is selected from the group consisting of methanol, ethanol, butanol, and mixtures thereof. In another particular embodiment, the solvent system is an alcohol (C₁-C₄):propionic (Hprop) acid mixture in a volume ratio range of from 20:80 to 50:50. In another particular embodiment, the solvent system is alcohol (C₁-C₄) propionic (Hprop) acid mixture in a volume ratio of 50:50. In another particular embodiment the solvent system Is methanol:Hprop in a volume ratio 50:50. In another particular embodiment the solvent system Is butanol:Hprop in a volume ratio 50:50. In another particular embodiment the solvent system Is ethanol:methanol:Hprop in a volume ratio 25:25:50. In another particular embodiment, the solvent system is ethanol:propionic (Hprop) acid mixture in a volume ratio range of from 20:80 to 50:50

In another particular embodiment, the amount of water in the solution is in a range of from 0.5 to 10% by weight. In another particular embodiment, the amount of water in the solution is lower than 2% by weight. In another particular embodiment, the amount of water in the solution is 0.6 wt %.

In a particular embodiment, the REBCO solution has a concentration of 1.75 M (sum of metals concentration) with a 4%v/v MEA for a Cu-rich mixture with the molar ratio of RE:Ba:Cu of 1:2:4.66; in a solvent mixture of 50:50 HProp:MeOH.

As mentioned above, any of the above embodiments with RE=Y is a particular embodiment of the invention.

In another particular embodiment of the present invention, the REBCO precursor solution is combined with nanoparticles. In another particular embodiment, the REBCO precursor solution of the present invention is that where the nanoparticles are BaMO₃ (M=Zr, Hf, Ti) nanoparticles, BaM₂O₆ (M=Ta, Nb) nanoparticles, or metal oxide nanoparticles. Thus, preformed nanoparticles can be added to the initial precursor solution to form nanocomposites that enhance superconductor performance at high magnetic fields.

All the features of the particular embodiments defined above for the precursor solution as product per se are also features of the corresponding particular embodiments of the process for its preparation or its uses according to the present invention.

The REBCO solutions of the present invention and in particular the YBCO solutions may be prepared by a process comprising the steps of: a) providing a mixture of (C₁-C₄) alcohol:propionic acid in a volume ratio in the range of from 20:80 to 60:40; b) adding barium propionate, copper propionate, and RE propionate to the mixture of solvents under stirring and in a consecutively manner, wherein each propionate salt is added once the previous propionate salt has been dissolved, and wherein the amount of propionate salts is an appropriate amount to obtain a total sum of metals concentration the propionate salts of 1-2 M; and c) adding an in a concentration between 1-8% in volume of the total volume of the solution, wherein the molar ratio amine:copper is in the range of form 0.3:1 to 2:1. It is a facile and fast method, which present endured stability and enable precise tunability of the final film thickness.

In a particular embodiment, the mixture of (C₁-C₄) alcohol:propanoic acid in a volume ratio in the range of from 20:80 to 50:50.

The REBCO precursor solutions may comprise further additives other than triethanolamine, i.e., it does not contain triethanolamine.

In a particular embodiment, the barium carbonate, copper oxide, and RE oxide used as starting materials have a purity equal to or higher than 99.9% by weight. In another particular embodiment, the barium carbonate, copper oxide, and RE oxide used as starting materials have a purity equal to or higher than 99.99% by weight. In another particular embodiment, the propionic acid has a purity equal to or higher than 99.5% by weight.

The three metal-propionate salts used in the present invention may be prepared through a facile one-pot syntheses of high purity and without hazard media that may hamper the successive preparation of high concentration YBCO precursor solutions.

No nitrogen media is used in the preparation of RE-propionate, in particular, Y-propionate, in order to avoid Ba(NO₃)₂ precipitation during the REBCO precursor solution synthesis.

The precursors of choice for the metal propionates were CuO, RE₂O₃, and BaCO₃, given their low cost and high purity commercially available. When the metal is Gd, then the precursor of choice is Gd₂O₃. Indeed, when compared to the respective acetate salts, a large cost-wise difference is observed, being the most notable the case of copper precursors where a cost decrease of at least a factor 10 is achieved.

The process for obtaining the propionate salts starting materials of the process of the present invention, is cost effective, robust and reproducible and allows preparing the three metal-propionate salts of high purity and without hazard media.

In a particular embodiment, the process for preparing the REBCO precursor solution as defined above, further comprising previously preparing each of the barium propionate, copper propionate, and RE propionate starting materials by reacting individually barium carbonate, copper (II) oxide, and yttrium (III) oxide respectively with an excess of propionic acid at an appropriate temperature and isolating each of the products thus obtained.

In another particular embodiment, the temperature at which each of the barium propionate, copper propionate, and RE propionate starting materials are prepared is comprised in a range from RT-150 °C. In another particular embodiment, the temperature is in a range of 120-140 °C. In another particular embodiment, the isolation of each one of barium propionate, copper propionate, and RE propionate comprises a washing procedure to eliminate the residual propionic acid with an organic solvent. In another particular embodiment, the solvent is a polar solvent such as diethyl ether or acetone. Advantageously, the compounds thus obtained are free of residual propionic acid.

As a way of example copper propionate (CuProp₂) can be prepared by a process comprising the following steps: a) adding copper (II) oxide with a purity higher than 99.9% by weight to an excess of propionic acid (≥99.5%) in a concentration around 0.3-1 M, in particular, 0.5 M; b) conducting the reaction under reflux; c) cooling down and eliminating the solvent excess, for instance, by filtration or by rotary evaporation to obtain a solid. A washing procedure can be carried out to eliminate residual solvent. The solid can be washed several times with a solvent, for instance, with diethyl ether. The solid can be grinded, for instance, using a ball milling with agate mortar (Fritsch, Pulverisette 6, 250 spheres of 5 mm diameter) to obtain a fine powder. The copper propionate obtained by the process described above may be characterized by ATR-FTIR spectroscopy, XRD; and SEM in the conditions described in the examples. Thermogravimetric Analysis (TGA) of the final product at a TG heating from 50 °C to 200 °C in Air at 10K/min indicates that the product is anhydrous, as there is no mass loss in the temperature range where hydration water molecules are lost (up to 140 °C). The copper propionate after the grinding process has a size between 1 μm and 20 μm measured by Scanning electron microscopy (SEM).

The barium propionate (BaProp₂) can be prepared by a process comprising the following steps: a) adding barium (II) carbonate (BaCO₃) with a purity higher than 99.9% to a mixture of propionic acid (≥99.5%) and distilled water, for example in a ratio 1.1:1, respectively, and mixing them at an appropriate temperature, for instance, room temperature (T 25 °C); b) eliminating the excess solvent and favouring the crystallization of the solid product by cooling below 0 °C, for instance, using a bath of ice and acetone. The solid can be washed several times to eliminate residual solvent, for instance, with acetone and/or diethyl ether. The solid can be grinded, for instance, using a ball milling with agate mortar as described above to obtain a fine powder. The product may be characterized by may be characterized by ATR-FTIR spectroscopy, XRD; and SEM in the conditions described in the examples. In addition, by nuclear magnetic resonance (NMR) spectroscopy ¹³C (CD₃OD) using a Bruker Avance DPX de 250 MHz (5.8 T) spectrometer can exclusively be seen the peaks due to the propionate species of Barium and indicate the absence of any undesired product. On the other hand, the TG heating process used from 50 °C to 200 °C in Air at 10K/min shows a TG curve evidencing that the product contains a 2% in mass due to H₂O and a 4% in mass due to propionic acid.

The yttrium propionate (YProp₃) may be prepared by a process comprising adding yttrium (III) oxide (99.99%), to an excess of propionic acid (≥99.5%), in a concentration 0.3-1 M, for instance, 0.5 M, reacting them under reflux; cooling down and elimination the solvent excess to obtain a white solid. A washing procedure may be carried out to eliminate residual solvent. The solid can be washed several times with a solvent such as acetone, and/or diethyl Ether. Finally, the solid can be grounded. The product may be characterized by ATR-FTIR spectroscopy, XRD; and SEM in the conditions described in the examples. ¹³C NMR spectra can be acquired using CD₃OD as solvent using a Bruker Avance DPX de 250 MHz (5.8 T) spectrometer. It shows exclusively the peaks due to the propionate species of Yttrium, thus indicating the absence of any undesired product. The TG heating process used was from 50 °C to 200 °C in Air at 10K/min. TG curve shows that the product contains a 6% in mass due to H₂O, and as the beginning of the dehydration is at 100 °C, this indicates that H₂O is coordinated to the structure. Through further TG-Analysis, it is seen that this quantity does not change over time.

Gadolinium Propionate (GdProp₃) may be prepared in the same way as yttrium Propionate. The solid obtained may be washed several times with diethyl ether. The product may be characterized by ATR-FTIR spectroscopy, XRD; and SEM in the conditions described in the examples. The heating process for the TG was from 50 °C to 200 °C in Air at 10 K/min The TG curve shows that the product contains a 6.2% in mass due to H₂O, and as most of the mass loss due to dehydration is above 100 °C, this indicates that H₂O is coordinated to the structure.

Simple addition of the three metal-propionate salts in a mixture of propionic acid and methanol (50:50) may yield a solution of 1 M-2 M in sum of salts as maximum concentration. In a particular embodiment, the concentration of the solution is 1 M.

The REBCO precursor colloidal solution with nanoparticles may be prepared by a process comprising: a) providing a solution of nanoparticles such as preformed metal oxide nanoparticles in an alcohol such as methanol or ethanol; b) adding ethanolamine to the solution; c) adding methanol and/or ethanol and then propionic acid; d) adding the salts, preferably in the following order: Barium propionate, Copper propionate, and Rare Earth metal propionate such as yttrium propionate. In a particular embodiment, the nanoparticles are initially dispersed in ethanol. Generally, the nanoparticle dispersion is in a molar concentration of nanoparticles appropriate to yield a molar concentration from 6 to 30% molar in the final REBCO precursor solution with nanoparticles. In a particular embodiment, the molar concentration of nanoparticles is that which yields a molar concentration from 8 to 20% molar, more particularly 8-12% molar, and even more particularly, 12% molar. All the ranges included herein include the endpoints of the range.

BaMO₃ (M=Zr, Hf, Ti) nanoparticles can be synthesized by using thermal activation methods such as autoclave and microwave with the following procedures, respectively. Both methods enable the synthesis of dispersed nanoparticles of controlled size. Nanoparticles size can be tuned in the range of interest, i.e. 4-5 nm, 7-8 nm, 10-12 nm.

As a way of example, the desired n-butoxide precursor (M =Ti⁴⁺, Zr⁴⁺ or Hf⁴⁺) can be added to ethanol at room temperature under N₂ atmosphere. Barium hydroxide octahydrate may be then added, followed by triethylene glycol and H₂O or ammonia depending on the nanoparticle (NP) size desired. Reagents may be added under continuous stirring at room temperature. The final suspension can be heated up at high temperature 100-180 °C in an autoclave and maintain at such temperature for an appropriate time, for instance, 1 hour, and then slowly cooled down to room temperature. The resulting suspension can be washed firstly, with an excess of ethyl acetate, then EtOH, MeOH, or BuOH, in particular, 1-butanol, can be added, and the mixture can be sonicated until total dispersion of the pellet. An extra centrifugation can be performed and repeated until homogenous size distribution is observed in TEM and DLS.

When using microwave activation, the process can be performed following the same procedure as solvothermal reaction using the desired amount of H₂O. After the mixing, the resulting solution can be heated with a heating ramp (5-40 °C/min) until 180 °C for the BaMO₃ (M=Zr⁴⁺, and Hf⁴⁺) and 100 °C for the BaTiO₃, holding the corresponding final temperature for an appropriate time such as 5-10 min. Afterwards, the same cleaning process as the solvothermal procedure can be performed and finally dispersed into absolute ethanol or methanol.

The BaM₂O₆ (M=Ta, Nb) nanoparticles can be prepared using an autoclave system able to reach high temperatures (up to 300 °C) and high pressure (from 3-14 bar) to perform a surfactant-free solvothermal reaction avoiding their agglomeration by a post-synthetic surface functionalization. This reaction can be carried out in an autoclave system. Mixed metal precursors in stochiometric ratio, Ta(OCH₂CH₃)₅ and Ba(OH)₂.8H₂O can be mixed at room temperature (20-25 °C) under N₂ atmosphere in for example EtOH. The solution can be heated up in an autoclave at high temperature (220-260 °C) during certain time (24-70 h). The resulting solution with precipitate can be washed with deionized water using centrifugation. Finally, the NPs can be redispersed in ethanol. A post-synthetic surface functionalization can be carried out by a process that comprises the redispersion of precipitated surfactant-free NPs in DMF in acetonitrile (MeCN) solution followed by the addition of Me₃OBF₄ in MeCN. The resulting nanoparticle solutions can be characterized by DLS, TEM, XRD and eventually by TGA, NMR and IR.

In a particular embodiment, the solution of nanoparticles in an alcohol is in a molar concentration such that in the final REBCO precursor colloidal solution are in a molar percentage with respect to the REBCO concentration of 8-30% molar. In another particular embodiment, such percentage is 8-20% molar, in another particular embodiment, such percentage is 6-18% molar. In another particular embodiment, such percentage is 6%, 12% or 18% molar.

In another particular embodiment of the present invention, the process for preparing the REBCO precursor solution is that wherein the mixture of propionic acid and methanol is in a volume ratio of 50:50.

In another particular embodiment of the present invention, the process for preparing the REBCO precursor solution is that wherein the amine is present in the range of from 4-6 vol/vol %. The use of the additive aid in obtaining homogeneous pyrolyzed layers of low porosity, which additionally is suitable for multi-deposition.

It is also part of the invention a process for preparing superconductors based on growing superconducting REBCO (or YBCO) layers from 0.1 μm to 2.5 μm thickness comprising the following steps: a) disposing the precursor solution as defined above onto a surface of a substrate by means of any method enabling the homogeneous control of the thickness of the film to form a precursor film; b) submitting the precursor film to a pyrolysis process by means of a thermal treatment in a controlled atmosphere; c) optionally repeating the steps a) to b) to obtain thicker pyrolyzed films by a multideposition process; d) submitting the deposited REBCO layer of step b) or of the REBCO multilayer of step c) to a process of TLAG for the growth of the final epitaxial REBCO layers; and e) submitting the superconductor thus obtained to an oxygenation process. Thus, this process combines deposition of a chemical solution with a TLAG growth process based on the decomposition and crystal growth of the deposited product.

In a particular embodiment, the process is for preparing superconductors based on growing superconducting REBCO (or YBCO) layers from 0.1 μm to 2.5 μm thickness. In another particular embodiment, the process is for preparing superconductors based on growing superconducting REBCO (or YBCO) layers from 0.2 μm to 1.5 μm thickness.

The substrate is preferably SrTiO₃ or a metallic substrate including biaxially textured oxide layers on top of the metallic substrate. Other substrates may also be suitable for the purposes of the present invention such as LaAlO₃, SrTiO₃, MgO. Buffered metallic substrates with manganites is the preferable option, but other compatible buffers with REBCO cell parameter are also feasible, as an example Cerium oxide, RE-doped Cerium oxide, MgO, Gd₂CuO₄ or other combinations.

By biaxially textured substrate is understood a metal substrate either already textured through a thermomechanical process and having one or several oxide buffer layers on top of it or a polycrystalline metallic substrate where biaxially textured oxide layers have been grown on top of it by any method.

In a particular embodiment, the process for preparing superconductors based on growing superconducting REBCO (including YBCO) layers from 0.1 to 2.5 μm thickness comprises the following steps: a) optionally, submitting a single crystal SrTiO₃ substrate to an annealing process at a temperature comprised between 800-950 °C for an appropriate time to obtain flat-terraced surfaces, followed by successively cleaning with acetone and methanol; b) disposing the precursor solution as defined above onto a surface of a the substrate via spin coating to form a precursor film; c) submitting the precursor film to a pyrolysis process by heating in humid oxygen flow up to 240 °C with an appropriate heating rate, then heating up to 500 °C with a an appropriate heating rate; d) followed by cooling to room temperature; e) optionally repeating the steps b) to d) to obtain thicker pyrolyzed films by a multideposition process; f) submitting the deposited REBCO layer of step d) or of the REBCO multilayer of step e) to a process of TLAG; and g) submitting the superconductor thus obtained to a oxygenation process. Advantageously, the TLAG-CSD technology provides a less expensive and higher throughput process, compared to the currently available to manufacture high critical current density superconducting films, this enables the production of high temperature superconductors at a commercially interesting price from the REBCO precursor solutions of the invention.

In a particular embodiment of the process, the single crystal SrTiO₃ substrate is submitted to an annealing process at a temperature in the range of from 850-900 °C. The time may be around 5 h.

In another particular embodiment, the precursor film is submitted to a pyrolysis process by heating in humid oxygen flow (0.01-0.24 L/min) up to 500 °C with a heating rate of 2-20 °C/min. In another particular embodiment, the precursor film is submitted to a pyrolysis process by heating in humid oxygen flow (0.12 L/min) up to 240 °C with a heating rate of 5 °C/min, then switching to a heating ramp of 3 °C/min up to 500 °C.

In a particular embodiment of the process, the pyrolyzed layers have a thickness in the range of 400-2700 nm.

The pyrolized samples may reach a thickness of 0.2-5 μm. For instance, the pyrolyzed samples with two layers result in 800 nm thick films, samples of four layers reach 1.5 μm in thickness, and most remarkably, samples of eight layers yield a final thickness of 2.7 μm,

In a particular embodiment, a homogeneous pyrolyzed film with a thickness from 0.2 to 5 μm through 8 layer multidepositions is prepared.

Thus, the REBCO solution of the present invention yield single layers of high thickness and is suitable for multideposition.

The precursor solution can be used for scalable deposition techniques such as ink jet printing and slot die coating. Inkjet printing is a CSD method, which relies on the deposition of chemical solutions in the form of drops in the range of picolitres (pL). The advantage relies on the production of thick films in one single deposition by tuning the printing parameters, which allows the homogenous depositions of large volumes of solution onto a substrate. As a way of example, for the present invention a Autodrop Professional System MD-P802, from MICRODROP Technologies, Ink Jet Printer equipped with 4 nozzles with independent electronics have been used. Slot die coating is a pre-metered coating deposition technique which allows obtaining long uniform layers from few nm to mm on different substrates. As a way of example, for the present invention metallic tapes with the following architecture: LSMO/epi-MgO/IBAD-MgO/Y₂O₃/Al₂O₃/Hastelloy C276 have been used. This protocol contains 3 steps: a) Cleaning of the tapes; b) Deposition by slot-die coating and drying; and c) Pyrolysis.

Layers prepared by using this solution, exhibit the desired precursor phases for TLAG: BaCO₃ (mainly in its orthorhombic phase), CuO and Y₂O₃, by XRD analyses (see FIG. 10). The REBCO solutions of the present invention provide smooth, crack-free films.

Preferably the superconducting material has the formula ReBa₂Cu₃O₇₋ₓ. It is characterized by having a critical temperature of 90 K and a current density between 2 and 4 MA/cm² at 77K.

Finally, it is part of the present invention a superconducting multilayer on an appropriate substrate, i.e., a coated conductor, obtainable by the process described above. The substrate may be as defined above, in particular a biaxially textured metallic substrate. The superconducting multilayer is a multideposited layer.

The produced coated conductor in a KM-length of high temperature superconductor layer deposited on a buffered metallic substrate can be used in many applications such as nuclear magnetic resonance, magnetic resonance imaging, wind turbines, power cables, fault current limiters, rotating machines for electrical aircrafts, high field magnets for fusion, accelerators among other, or superconducting magnetic energy storage devices.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim and shall not be construed as limiting the scope of the claim. Furthermore, the present invention covers all possible combinations of particular and preferred embodiments described herein.

### EXAMPLES

### Example 1: Synthesis of Metal Propionates Precursors

Example 1A: Preparation of (Cu(Prop)₂): CuO (copper (II) oxide, Puratronic®, 99.7% (metals basis) purchased from Alfa Aesar) is added to an excess of Propionic Acid (HProp) (≥99.5%, purchased from Sigma Aldrich), for a total concentration of 0.5 M. The reagent and the solvent are used without any further purification. The reaction was conducted overnight at 140 °C with a reflux under vigorous stirring until a clear, blue solution was achieved. Excess solvent was eliminated using a Rotary Evaporator (Büchi), to obtain a dry, dark blue solid of copper propionate (Cu(Prop)₂).
Example 1B: Preparation of Y(Prop)₃: Y₂O₃ (yttrium (III) oxide, Reacton®, 99.99% (REO), purchased from Alfa Aesar) is added to an excess of HProp, to reach a concentration of 0.5 M. The reagent and the solvent are used without any further purification. The reaction is conducted overnight at 140 °C with a reflux under vigorous stirring until a transparent, clear solution was achieved. Excess solvent was eliminated using a Rotary Evaporator (Büchi), to obtain a dry, white solid of yttrium propionate (Y(Prop)₃).
Example 1C: Preparation of Ba(Prop)₂: The synthesis of barium propionate (Ba(Prop)₂) differs from the former syntheses, with BaCO₃ (Barium (II) carbonate, 99.95% (metals basis), purchased from Alfa Aesar) added to a mixture of HProp and distilled water, in ratio 1.125:1. The reagent and solvents are used without further purification. The reaction starts as a highly foamy, white solution and is stirred vigorously during 24 h, Excess solvent was eliminated using a Rotary Evaporator (Büchi), to obtain a transparent gel. To induce crystallization of the solid product from the gel, a bath of ice and acetone for 4 h was applied. Finally, all three solid products were washed with diethyl ether (Et₂O, AGR, ACS, ISO, stabilized with BHT, purchased from Labbox) using a Büchner Funnel, a necessary step to eliminate any residual HProp, resulted in high purity powder products; through this synthetic method we ensure yields of above 90%.

For Ba(Prop)₂ and Y(Prop)₃, a two-steps washing procedure was implemented, starting from Acetone (Acetone, Multisolvent® HPLC grade ACS ISO UV-VIS), necessary to improve the process of gel removal, and then followed by Et₂O. The yields increase from 20% to 90% and from 60% to 90% for the Ba-prop and Y-prop, respectively.

However, in case of Cu(Prop)₂, the as synthesized final powder product presented a large grain size, with the majority of grains over 100 μm; moreover, the grains showed a crystal-like shape, hindering its complete solubility in the following REBCO precursor solution for high concentrations. Therefore, the product was grinded, using a Fritsch Pulverisette 6 mono-planetary ball milling, adding to each of the two 80 mL agate bowls 14 g of product and 250 agate balls of 5 mm diameter. Given the high energetic force of the process, only 1 minute of grinding at 650 rpm was sufficient to importantly decrease grain size, with majority of grains after grinding being of a diameter of ~30 μm.

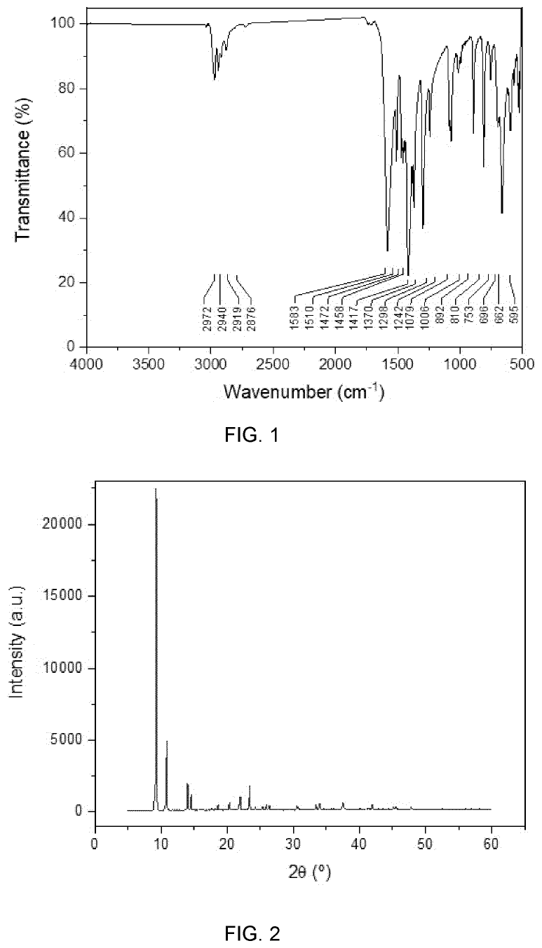

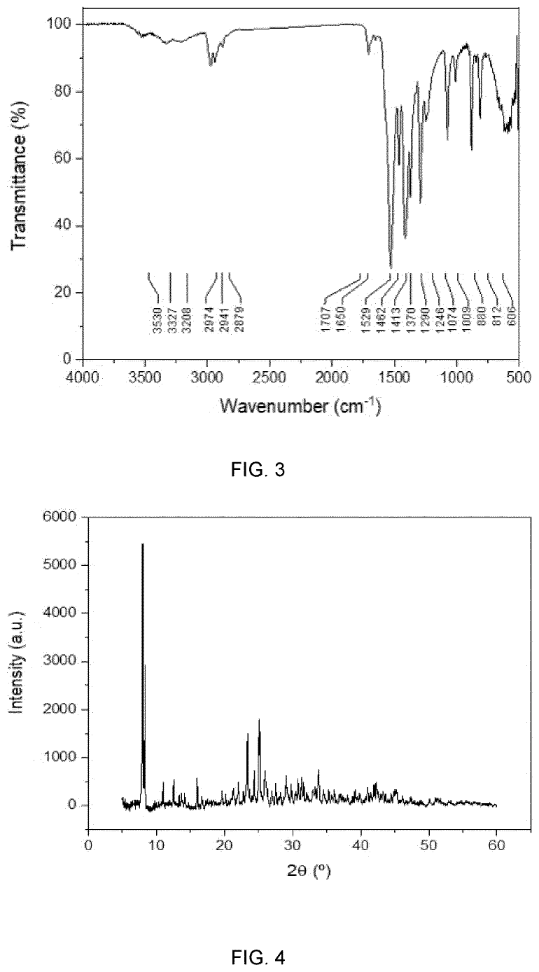

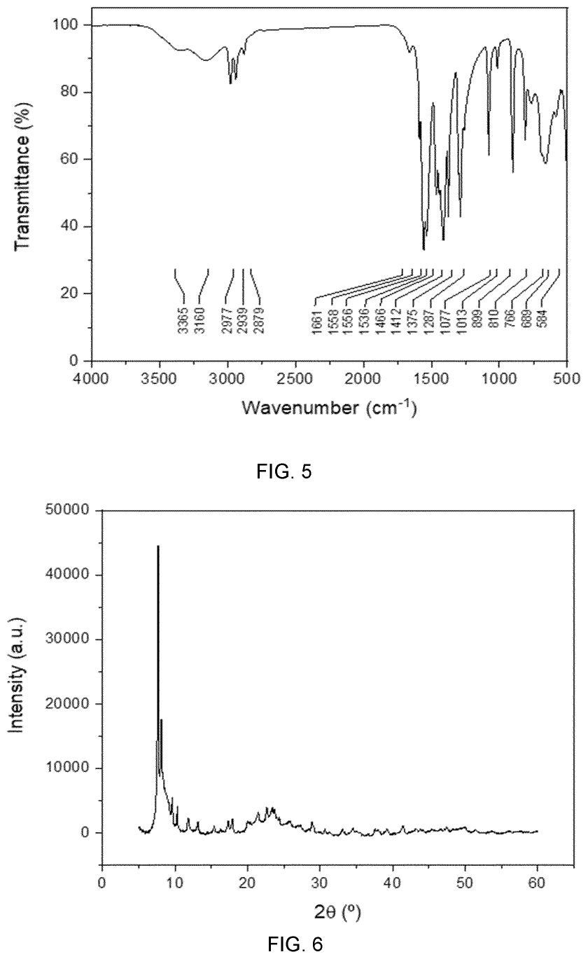

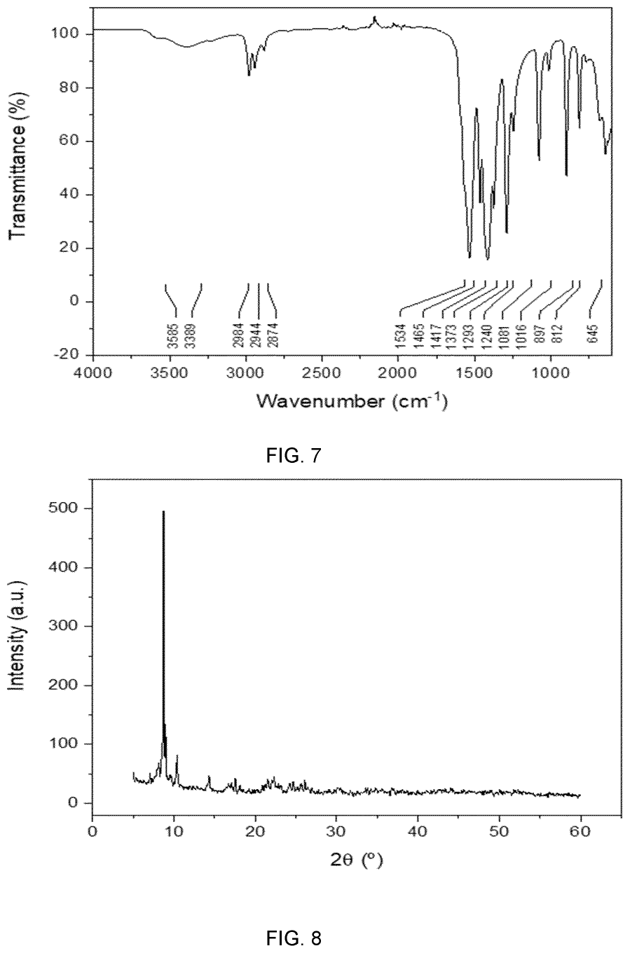

The final products are characterized by ATR FT-IR Spectroscopy (Spectrophotometer Jasco 4700, Energy range: 300-7800 cm−1, equipped with Attenuated Total Reflectance accessory) (FIGS. 1, 3, 5 and 7). The three peaks in the range 2980-2870 cm−1 are characteristic of the aliphatic chain of the propionate group, present in the three spectra. They may also be characterized by its XRD pattern which shows the characteristic peaks of the propionate salts (FIGS. 2, 4, 6, and 8). SEM (QUANTA FEI 200 FEG-ESEM) was used for the evaluation of grain size. The copper propionate after the grinding process has a grain size of diameter between 1 μm and 20 μm measured by Scanning electron microscopy (SEM). The BaProp₂ after the grinding process has a grainsize between 2 μm and 6 μm. The YProp₃ powder after grinding has a grain size between 5 μm and 50 μm.

The GdProp₃ powder after grinding has a grain size are between 5 μm and 80 μm. NMR Spectrometry (Bruker Advance DPX, 250 MHz (5.8 T), characterization restricted to Ba(Prop)₂ and Y(Prop)₃, as due to the paramagnetic nature of Cu(II) in Cu(Prop)₂.

### Example 2: Preparation of YBCO Precursor Solution

The preparation of YBCO precursor solutions follows the same procedure independently of the stoichiometries of the solution in question. The various stoichiometries of solutions differ in the Y—Ba—Cu ratio, and are named considering the Ba-Cu molar ratio of the transient liquid formed during the transient liquid assisted growth (TLAG) process, as following: the YBCO-stoichiometric mixture with a Y—Ba—Cu molar ratio of 1:2:3 ((2:3) composition); a Cu-rich mixture with molar ratio of Y—Ba—Cu of 1:2:4.66 ((3:7) composition); excess of Cu-rich, composition corresponding to a molar ratio of Y—Ba—Cu of 1:2:5.5 ((4:11) composition) and Y-rich Y—Ba—Cu of 1.35:2:5.5 (Y-rich 4:11). These solutions were prepared using as solvents a mixture in the volume ratio (50:50) of HProp and methanol (MeOH) (methanol, 99.9%, anhydrous (max. 0.003% H₂O), purchased from Scharlab), and the addition of different % v/v of ethanolamine (MEA) (Ethanolamine, purified by redistillation, ≥99.5%, purchased from Sigma Aldrich). The concentration of YBCO solutions for spin-coating deposition was 1.75 M in sum of metal salts when MEA is used, independently of the solution composition. MEA aids complete precursors dissolution increases stability of solutions and increases homogeneity and thickness of the final films. If no amine additive is used, only a total concentration of 1 M in sum of salts could be solubilized. The metal propionates precursor salts were added to the mixture of solvents in consecutive order, allowing for the complete dissolution of each precursor in the solution before the addition of the following one. Ba(Prop)₂ was the first to be added to the mixture of solvents, showing a fast dissolution, with the solution to appear transparent once dissolution is completed. Cu(Prop)₂ was then added and heated at 30 °C Subsequently, Y(Prop)₃ was added. The solution was left at 30 °C and 450 rpm for 30 more minutes. Complete dissolution of all the precursors was achieved yielding an intense blue coloured solution. The solvents mixture was adjusted to 50:50 volume of solvents (as previously specified) and filtered. The final solution was stored in a sealed vial with Ar atmosphere.

Solutions' rheological properties such as Viscosity and Contact angle were measured with a HAAKE RheoStress RS600 from Thermo Electron Corp and Drop Shape Analyzer DSA 100 from Krüss, respectively (Table S2†). Water content of the solutions is crucial, as it may influence the final properties of the REBCO layers; it is thus monitored through the Karl Fischer Titration Method (Ref.) (Nittoseiko Analytech, Model CA-310 equipped with VA-200 vaporizer) and each solution is only used until % H₂O<2 wt %. Electron Paramagnetic Resonance (Bruker ELEXYS E500 X band EPR spectrometer) measurements were carried out on certain solutions to examine and explain the role of MEA (See Results Section).

### Viscosity

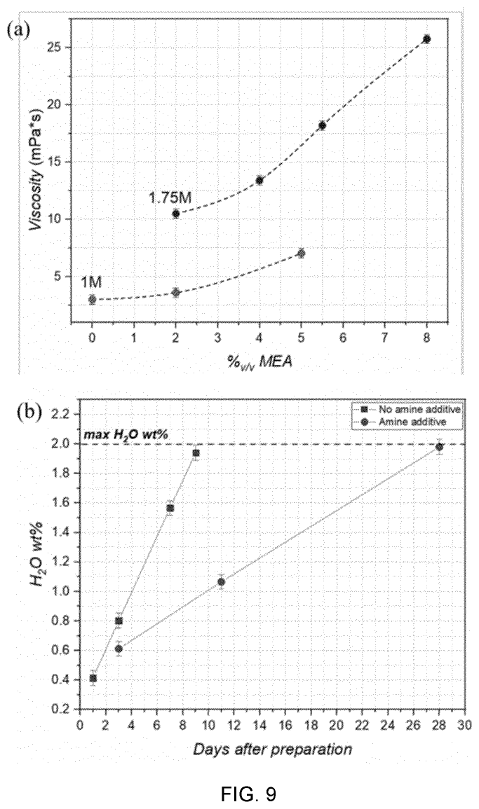

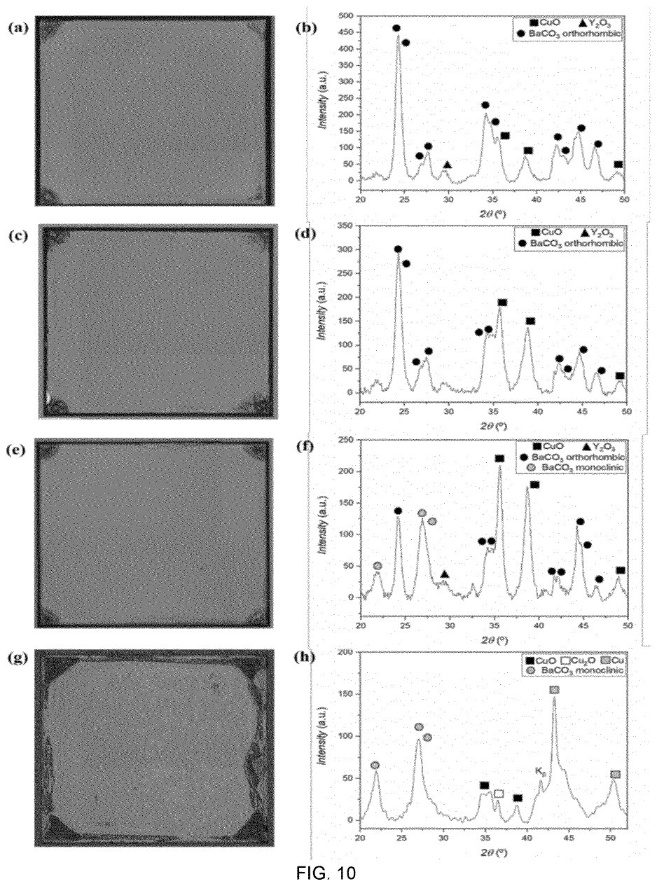

FIG. 9a displays a series of viscosity measurements conducted on solutions of two concentrations, 1 M and 1.75 M in sum of metals. The addition of MEA is of great benefit to the purpose of increasing final thickness.

### Water Content

As this class of solutions uses a mixture of HProp and MeOH (50:50) as solvents, they are y subject to Fischer esterification, producing H₂O as by-product of this reaction. FIG. 9b shows the H₂O wt % evolution of two (3:7) composition solutions over time.

A complex with the MEA is formed. Being the amine (MEA) not in stoichiometric quantity with the Cu(Prop)₂, it gives a reason for the concomitant existence of two species in solution. The complex Cu-MEA is more stable in solution than the Cu(Prop)₂ alone.

The formation of the complex is seen by EPR.

### Example 3: Thin Film Deposition, Pyrolysis and Growth

Thin films with different compositions were deposited via spin coating (SMA 6000 Pro, purchased from Suministro de Materiales y Asistencia, S.L.) using as-prepared YBCO precursor solutions.

Single-crystal (001) SrTiO₃ (STO) substrates (CrysTech GmbH) were used, and before deposition, these undergo an annealing process at 900 °C for 5 h to obtain flat-terraced surfaces, successively cleaned with Acetone (Acetone, Multisolvent® HPLC grade ACS ISO UV-VIS, purchased from Scharlab), and Methanol (Methanol, Multisolvent® HPLC grade ACS ISO UV-VIS K.F., purchased from Scharlab), to eliminate any possible residues. The solution was deposited in a grade ISO7 clean room 10% humidity at a spinning rate of 6000 rpm for 2 min. The pyrolysis process was done by heating in humid oxygen flow (0.12 L/min) up to 240 °C with a heating rate of 5 °C/min, then switching to a heating ramp of 3 °C/min up to 500 °C, followed by cooling to room temperature. To obtain thicker pyrolyzed films, multideposition processes were carried out repeating the above procedure. Finally, the growth of the final epitaxial YBCO layers through the innovative process of TLAG was improved using a tubular furnace connected to a vacuum system. Transient Liquid Assisted Growth (TLAG) Process through PO2—route experiments were carried out in a tubular furnace equipped with a vacuum system that enabled to switch fast (~seconds) from a low vacuum to a high vacuum. The PO₂ was controlled by introducing gas lines in the vacuum system with regulating valves. The samples were heated at low PO₂(10⁻⁵-10⁻⁶ bar), with an average heating rate of 1 °C/s to the desired temperature. The jump in Pₒ₂ followed, reaching the desired final Pₒ₂ in a time range of 1 s. Cooling of the samples was performed with the same heating rate. Following the TLAG process, an oxygenation process was performed. The samples were heated in a tubular furnace at 1 bar, with a heating rate of 10 °C/min to 450 °C, followed by a dwell of 210 min, then cooling with the same heating rate to RT. This process was performed under a continuous O₂ flow.

### Sample Characterization

Characterization of pyrolyzed samples was performed using a variety of techniques, starting from an optical microscopy (OM) (Leica DM1750 M) analysis, to inspect homogeneity of the films, as well as reflectometry measurements (Filmetrics F50) to obtain the thickness of the film through a rapid and non-destructive technique. The results for different samples can be seen in FIG. 10 (a, c, e, g).

### Structural Characterisation

Structure and phase composition of the as-prepared pyrolyzed layers were characterized using XRD on a Bruker-AXS D8 Advance diffractometer (Cu K_{α} equipped with general area detector diffraction system (GADDS)). For the grown YBCO thin films, XRD characterization sees the use of both a Bruker-AXS D8 Advance diffractometer (Cu Kα) equipped with general area detector diffraction system (GADDS) and a Bruker D8 Discover system (Cu Kα, X-ray energy=8.049 keV) equipped with a Lynxeye XE-T energy-dispersive one-dimensional (1D) detector, measuring in two configurations: 0-2θ geometry to evaluate epitaxy, and grazing incidence (GI) geometry to amplify signals coming from secondary phases, facilitating their identification.

The XRD analyses of layers prepared by using this solution, shown in FIG. 10 (b, d, f, h), exhibit the desired precursor phases for TLAG: BaCO₃ (mainly in its orthorhombic phase), CuO and Y₂O₃, confirming this class of solutions for the growth of thick and robust YBCO films. And that the precursor solution according to the invention can provide smooth, crack-free films for all three compositions, i.e., (2:3), (3:7), and (4:11).

### Microstructural Characterization

Surface morphology was evaluated using Scanning electron microscopy (SEM) through the use of a QUANTA FEI 200 FEG-ESEM together with Energy-dispersive X-ray (EDX) spectroscopy.

For thickness evaluation, distribution, and sizes of nanocrystalline phases, the microstructure of pyrolyzed thin films the following techniques were used: a) high-resolution transmission electron microscopy (HRTEM), b) high-angle annular dark field scanning transmission electron microscopy (STEM-HAADF), c) energy dispersive X-ray spectroscopy (EDX), and d) electron energy loss spectroscopy (EELS). For this purpose, FEI Tecnai F20 (S)TEM operated in both TEM and STEM mode at 200 kV, equipped with a Gatan quantum electron energy-loss spectrometer for EELS analyses, was used.

The pore density was evaluated from the cross-sectional STEM-HAADF micrographs. The images were processed with an image analysis software ImageJ41 where well-defined dark contrast areas were defined as pores using the threshold tool and analysed with the ‘analyze particle function’. The average pore sizes were calculated from the histograms of measured pore areas considering round pores.

For EELS data processing, principal component analysis (PCA) was used in order to reduce the statistical noise in EELS spectrum images. A reconstruction using the first 10 principal components was performed using the weighted-PCA multivariate statistical analysis (MSA) Plugin42 within Gatan Digital Micrograph software.

Moreover, the microstructure, atomic-defect structure, and phase composition of grown YBCO thin films were studied using FEI Tecnai F20 (S)TEM operated at 200 kV, as well as FEI Titan with a X-FEG gun, a CESCOR Cs-probe corrector and a Gatan TRIDIEM 866 ERS energy filter with a monochromator, operated in STEM mode at 300 kV.

For transmission electron microscopy (TEM), cross-sectional specimens were prepared by conventional methods: cutting, gluing the slices face-to-face, thinning down by tripod mechanical polishing, followed by Ar+ ion milling using Gatan PIPS, until electron transparency was attained.

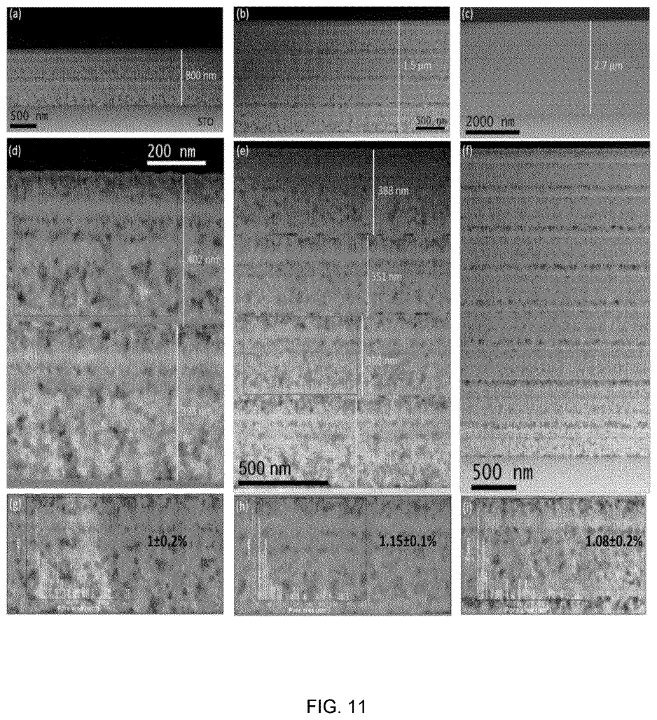

The cross-sectional high-angle annular dark field scanning transmission electron microscopy (STEM-HAADF) images display that the pyrolyzed samples with two layers result in 800 nm thick films, samples of four layers reach 1.5 μm in thickness, and most remarkably, samples of eight layers yield a final thickness of 2.7 μm, implying no loss of homogeneity or differences in the nanocrystalline matrix (FIG. 10). In detail, the low magnification STEM-HAADF images exhibit the smooth surfaces for all three (3:7) pyrolyzed thin films along with their respective thicknesses (FIG. 11 (a-c)). Moreover, FIG. 11 (d-f) reveals that CuO nanocrystals are homogeneously distributed within a BaCO₃ matrix, with no segregations or interfaces, ideal situation for a successful epitaxial growth of YBCO. In fact, segregated CuO interlayers at the interfaces of different layers in multiple depositions with intermediate pyrolysis, are a common downside, especially widespread in TFA-route. In FIG. 11(g-i), through the STEM-HAADF image analysis using the software ImageJ, estimated pore densities as low as 1±0.2%, 1.15±0.1%, and 1.08±0.2% are determined for pyrolyzed films with two, four, and eight layers, respectively.

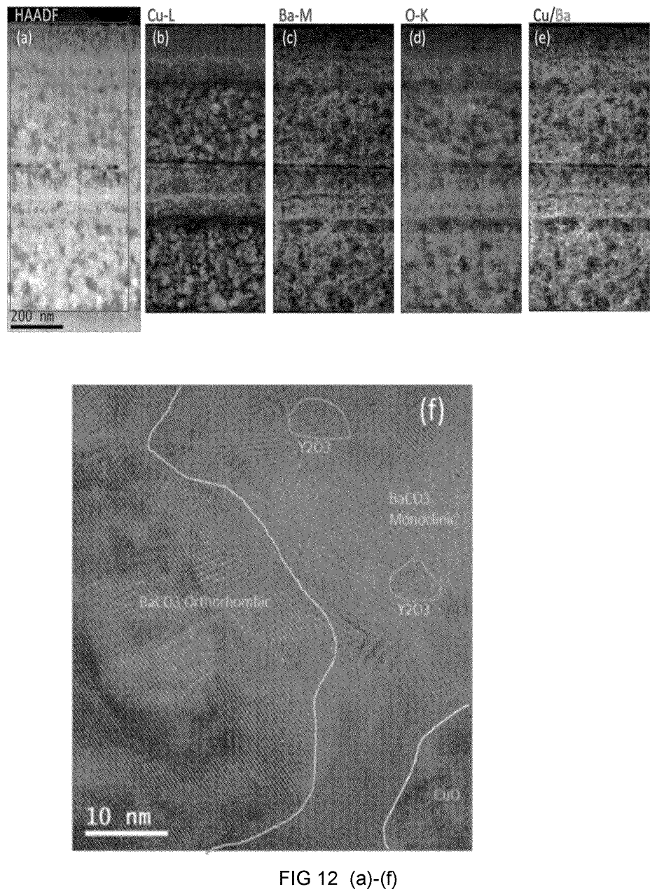

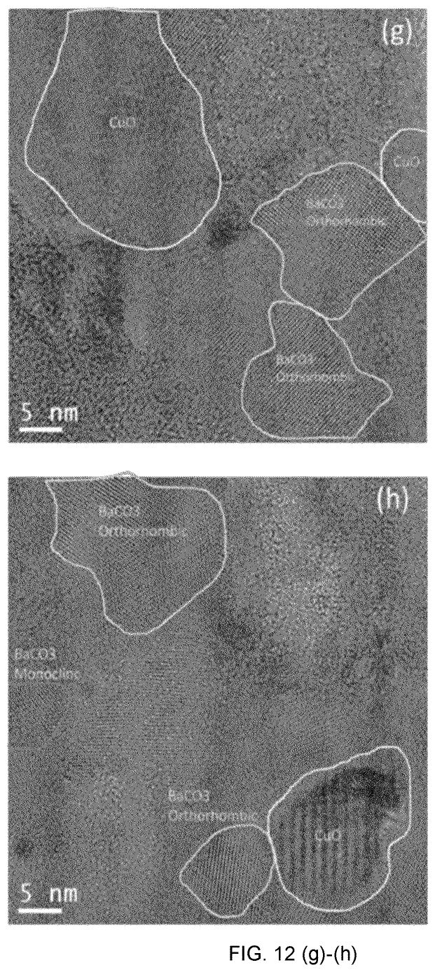

Further elemental analysis on these pyrolyzed films was performed through electron energy loss spectroscopy (EELS), where elemental maps of CuL_{2,3}, BaM_{4,5}, and OK, reveal high homogeneity of these precursor phases at the nanoscale (FIG. 12 a-e).

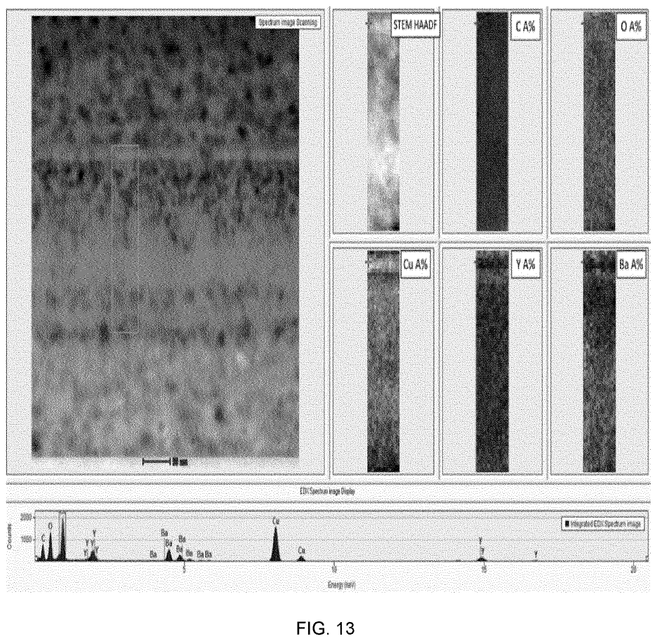

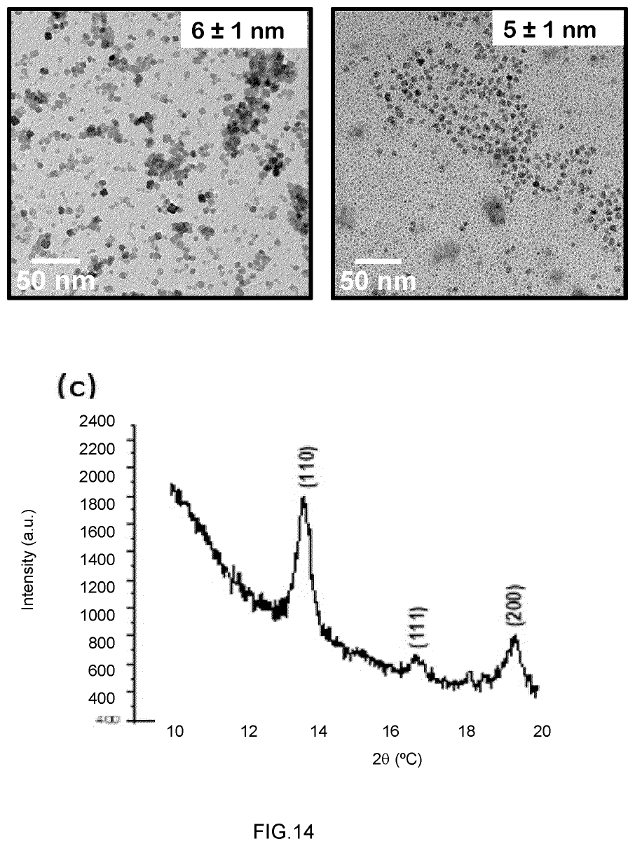

A complementary compositional analysis is conducted availing energy dispersive X-ray spectroscopy (EDX) (FIG. 13), and Y, Ba, Cu STEM-EDX cross-sectional elemental maps also confirm the uniform distribution of nanocrystalline phases in a (3:7) composition pyrolyzed thin film deposited using 1.75 M+4% v/v MEA solution.

The spatial distribution, crystalline state, and sizes of precursor phases could be determined through the analysis of high-resolution transmission electron microscopy (HRTEM) images.

All three nanocrystalline phases, i.e., BaCO₃, CuO, and Y₂O₃, are distinctly identified in multiple HR-TEM images corroborating a robust solution composed by these precursor intermediates, with typical diameters of 10-30 nm for orthorhombic BaCO₃, 5-7 nm for monoclinic BaCO₃, 10-25 nm for CuO, and Y₂O₃ remain as small as 5-6 nm (FIG. 12f-h). Being TLAG an ultrafast liquid-assisted process, the small size and homogeneous distribution of the nanocrystalline precursors greatly favour homogenous and fast liquid formation by easing atomic mobility, promoting high epitaxial layers at ultrafast growth rates. Likewise, as the majority of BaCO₃ is present in the orthorhombic phase, it is advantageous due to its straightforward reaction with nanocrystalline CuO in the following TLAG process.

### Electrical Characterization

The self-field critical current density (Jc) values were obtained from out-of-plane inductive measurements in a commercial Quantum Design MPMS XL SQUID DC magnetometer equipped with a 7 T magnet. Jc self-field (at 5 K and 77 K) was deduced from the remanent magnetization width of the hysteresis loop by applying the using the Bean critical state model for a thin disk. A Quantum Design Physical property measurement system (PPMS) device has been used for resistivity measurements through the Van der Pauw configuration and Tc determination. Also, the same PPMS was used to determine the I(V) characteristics and the transport critical current density with an electric field criterium of 1 μV/cm.

The values obtained for the YBCO superconducting layers of thickness beyond 500 nm were of a critical current density between 2-4 MA/cm² at 77K and 20-30 MA/cm² at 5K, both self-field, and a critical temperature of 90 K.

### Example 4: Preparation of REBCO Precursor Colloidal Solution With Metal-Oxide Nanoparticles

For the synthesis of REBCO precursor solution using preformed metal-oxide nanoparticles (from 5-10 nm of size) and the different REBCO stoichiometries used as Y:Ba:Cu 1:2:4.66 (3-7) composition, Y:Ba:Cu 1:2:3 (2-3) composition and other compositions we used two methodologies with the following steps:

For High REBCO total Metal Concentrations (1-1.5 M)

In a round bottom flask 1-4% v/v of monoethanolamine (MEA) were added depending on the total metal concentration desired (1-1.5 M). The % NP mol (6-24% mol) desired were added to the flask at room temperature under inert atmosphere (Ar) and stirring for 5 min. A 50% of the final desired volume with ethanol or/and methanol were added. (For example, 50% ethanol or 50% methanol or 25% ethanol and 25% methanol). A 50% of total final volume of propionic acid was added. The solution was stirred until complete NP redispersion,

After NP redispersion, Barium propionate, and Copper propionate were added. The temperature was kept between 30 to 50 °C depending on the total metal concentration under Ar flow and stirring until complete dissolution of salts (30 min-2 h).

Yttrium was added under Ar flow again and then Ar flow was closed and left under stirring until complete dissolution (30 min-1 h). After complete dissolution (clear blue solution) of salts, the reaction was cooled down to room temperature under stirring. 9) The solution was transferred to a volumetric flask and to the appropriate volume with the same solvent mixture used before. Finally, the solution was filtered with a specific filter for viscous solutions and transfer to a vial, sealed under Ar and stored in a desiccator.

### For Low REBCO Total Metal Concentrations (0.5-0.875 M)

Prepare a REBCO precursor solution without nanoparticle addition following the same procedure described in section 1.2 with the desired total metal concentration (1-1.75 M). Mix 50% of REBCO precursor solution without nanoparticle prepared in step 1 with 50% of nanoparticle solution with the desired % NP mol (NP were redispersed in 100% methanol or 100% ethanol or a mixture of methanol/ethanol). Viscosity and water content of REBCO nanocomposite precursor solutions were eventually characterized. Moreover, NP stability in REBCO nanocomposite precursor solution was checked sometimes by TEM and solution XRD.
Viscosity: 11.8 MPa·s
H₂O Content: 1.1% (1^{st} Day of Synthesis)

In TEM images it is possible to check the nanoparticle stability in YBCO nanocomposite precursor solution as the nanoparticle size and distribution similar as when we compare with the nanoparticle redispersed in only ethanol. Nanoparticles crystallinity and stability in YBCO precursor solvents and in the presence of the additive MEA was confirmed also by solution XRD.

### CITATION LIST

### Patent Literature

CN106242553A

### Non-Patent Literature

L. Soler et al.; Nat. Commun., 2020, vol. 11, p.344
P. Vermeir et al.; “Elucidation of the Mechanism in Fluorine-Free prepared YBa₂Cu₃O_{7-δ} Coatings” Inorg. Chem., 2010, vol. 49, pp. 4471-4477
P. Vermeir et al., “Influence of sintering conditions in the preparation of acetate-based fluorine-free CSD YBCO films using a direct sintering method”; Mater. Res. Bull., 2012, vol. 47, pp. 4376-4382
Yue Zhao et al; “Growth of Highly Epitaxial YBa2Cu3O7-δ Films from a Simple Propionate-Based Solution”, Inorg. Chem. 2015, vol. 54, pp. 10232-10238
S. Rasi et al.; “Relevance of the Formation of Intermediate Non-Equilibrium Phases in YBaCuO im Growth by Transient Liquid Assisted Growth”; The Journal of Physical Chemistry 2020 vol. 124; pp. 15574-15584
M. Nasui et al; “Fluorine-fee propionate route for the chemical solution deposition of YBa2Cu3O7-x superconducting films”; Ceramics International 2015, vol. 41, pp. 4416-4421

What is claimed is:

1. A REBCO precursor solution comprising:
A propionate salt of Re;
A propionate salt of Ba;
A propionate salt of Cu;
An amine; and
A solvent system;
wherein:
RE means Y or earth rare metal;
the metals are RE, Ba, and Cu are found exclusively in the form of propionate salts; and the propionate salts are the only salts present in the solution;
the metals are in a total metal molar concentration in the range between 1-2 M of the solution;
the solvent system is an alcohol (C₁-C₄):propionic acid mixture in a volume ratio of 20:80-60:40;
the amine is an amine which is miscible in the solvent system at room temperature and is present in a concentration between 1-8% in volume of the total volume of the solution;
with the molar ratio amine:copper is of 0.3:1-2:1; and
the solution is free of F and free of acetate.

2. The REBCO precursor solution according to claim 1, wherein the solvent system is an alcohol (C₁-C₄):propionic acid mixture in a volume ratio of 20:80-50:50;

3. The REBCO precursor solution according to claim 1, wherein the amine is selected from the group consisting of (C₁-C₄)-alcoholamine, catecholamine, triethylamine, lutidine, 4-formylpyridine, 2-acetylpyridine, and pyridine.

4. The REBCO precursor solution according to claim 1, wherein the concentration of the amine in the solution is between 1-4% in volume in respect to the total volume of the solution.

5. The REBCO precursor solution according to claim 1, wherein the molar ratio amine:copper is 0.6:1

6. The REBCO precursor solution according to claim 1, wherein the solvent system is selected from the group consisting of: methanol:Hprop in a volume ratio 50:50, butanol:Hprop in a volume ratio 50:50, ethanol:methanol:Hprop in a volume ratio 25:25:50; and ethanol:Hprop in a volume ratio range of from 20:80 to 50:50.

7. The REBCO precursor solution according to claim 1, in combination with nanoparticles.

8. The REBCO precursor solution according to claim 1, which is selected from the group consisting of
a) a REBCO-stoichiometric mixture with a RE-Ba—Cu molar ratio of 1:2:3;
b) a Cu-rich mixture with molar ratio of RE-Ba—Cu of 1:2:4.66;
c) an excess Cu-rich mixture with a molar ratio of RE-Ba—Cu of 1:2:5.5; and
d) a RE-rich and excess of Cu-rich mixture with a molar ratio of RE-Ba—Cu of 1.35:2:5.5.

9. A process for preparing the REBCO precursor solution according to claim 1, wherein:
when the REBCO precursor solution is absence of nanoparticles, the process comprising the steps of:
a) providing a mixture of (C₁-C₄) alcohol:propionic acid in a volume ratio in the range of from 20:80 to 60:40;
b) adding, barium propionate, copper propionate, and RE propionate to the mixtures of solvents in a consecutively manner, wherein each propionate salt is added once the previous propionate salt has been dissolved, and wherein the amount of propionate salts is an appropriate amount to obtain a total sum of metal concentration of the propionate salts in a range of from 1 to 2 M; and
c) adding an amine in a range of from 1-8 vol/vol %, wherein the molar ratio amine:copper is in the range of form 0.3:1 to 2:1;
when the REBCO precursor solution comprises nanoparticles, the process comprises the steps of:
a) providing a solution of nanoparticles such as preformed metal oxide nanoparticles in an alcohol such as methanol or ethanol;
b) adding ethanolamine to the solution;
c) adding methanol and/or ethanol and then propionic acid; and
d) adding the salts in the following order: barium propionate, copper propionate, and RE propionate.

10. The process for preparing the REBCO precursor solution according to claim 9, wherein the amine is present in the range of from 4-6 vol/vol %.

11. The process for preparing the REBCO precursor solution according to claim 10, wherein the barium propionate, copper propionate, and RE propionate used as starting materials have a purity equal to or higher than 99.9% by weight.

12. The process for preparing the REBCO precursor solution according to claim 9, further comprising previously preparing each of the barium propionate, copper propionate, and RE propionate starting materials by reacting individually barium carbonate, copper (II) oxide, and yttrium (III) oxide respectively with an excess of propionic acid at an appropriate temperature and isolating each of the products thus obtained.

13. A process for the preparation of superconducting REBCO layers having a thickness of from 0.1 μm to 2.5 μm, comprising using the REBCO precursor solution as defined in claim 1.

14. The process according to claim 13, comprising the following steps:
a) disposing the precursor solution onto a surface of a substrate by means of any method enabling the homogeneous control of the thickness of the film to form a precursor film;
b) submitting the precursor film to a pyrolysis process by means of a thermal treatment in a controlled atmosphere;
c) optionally repeating the steps a) to b) to obtain thicker pyrolyzed films by a multideposition process;
d) submitting the deposited REBCO layer of step b) or of the REBCO multilayer of step c) to a process of TLAG for the growth of the final epitaxial REBCO layers; and
e) submitting the superconductor thus obtained to an oxygenation process.

15. Multideposited superconducting layer on an appropriate substrate obtainable by the process of claim 14.

16. The REBCO precursor solution according to claim 2, wherein the amine is selected from the group consisting of (C₁-C₄)-alcoholamine, catecholamine, triethylamine, lutidine, 4-formylpyridine, 2-acetylpyridine, and pyridine.

17. The REBCO precursor solution according to claim 16, wherein the concentration of the amine in the solution is between 1-4% in volume with respect to the total volume of the solution.

18. The REBCO precursor solution according to claim 17, wherein the molar ratio amine:copper is 0.6:1.

19. The REBCO precursor solution according to claim 18, wherein the solvent system is selected from the group consisting of: methanol:Hprop in a volume ratio 50:50, butanol:Hprop in a volume ratio 50:50, ethanol:methanol:Hprop in a volume ratio 25:25:50; and ethanol:Hprop in a volume ratio range of from 20:80 to 50:50.

20. The REBCO precursor solution according to claim 19, in combination with nanoparticles.
